(19) United States
(12) Patent Application Publication
(10) Pub. No.: US 2010/0198124 A1
(43) Pub. Date: Aug. 5, 2010

(54) SYSTEM AND METHOD FOR CONTROLLING THE JOINT MOTION OF A USER BASED ON A MEASURED PHYSIOLOGICAL PROPERTY

(76) Inventor: Kern Bhugra, Moffett Field, CA (US)

Correspondence Address: SCHOX PLC, 500 3rd Street, Suite 515, San Francisco, CA, 94107, US

(21) Appl. No.: 12/363,567

(22) Filed: Jan. 30, 2009

(51) Int. Cl. A61F 5/00; A61B 5/103; A61B 5/00

(52) U.S. Cl. 602/5; 600/587; 600/301

(57) ABSTRACT

In one embodiment, the apparatus for controlling the motion of a joint of a user includes a physiological sensor that measures a physiological property of the user, an orthosis that controls the motion of the joint of the user, an orthosis sensor that monitors a physical property of the orthosis, and a processing unit that processes data from the physiological sensor and from the orthosis sensor. In another embodiment, the method for controlling the motion of a joint of a user includes measuring a physiological property of the user with a physiological sensor, controlling motion of the joint of the user with an orthosis, monitoring a physical property of the orthosis with an orthosis sensor, and processing data from the physiological sensor and from the orthosis sensor.

### TECHNICAL FIELD

This invention relates generally to the orthosis field, and more specifically to an improved system and method for controlling the joint motion of a user that incorporates a measured physiological property.

### BRIEF DESCRIPTION OF THE FIGURES

FIG. 1 is a schematic representation of a preferred system of the invention.

FIG. 2 is a flowchart diagram of a preferred method of the invention.

FIG. 3 is a schematic diagram of a variation of the preferred system of the invention.

### DESCRIPTION OF THE PREFERRED EMBODIMENTS

The following description of the preferred embodiments of the invention is not intended to limit the invention to these preferred embodiments, but rather to enable any person skilled in the art to make and use this invention.

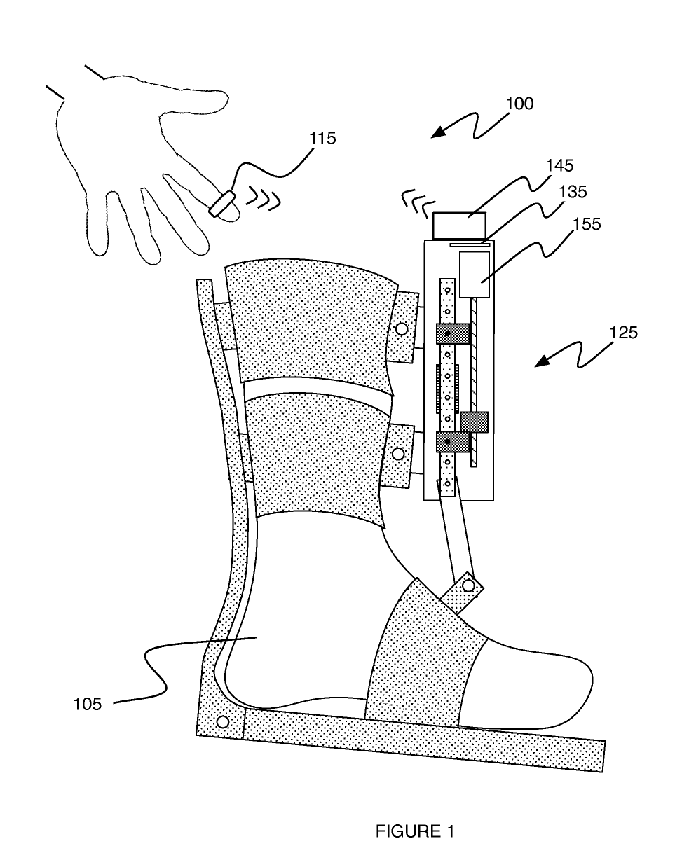

As shown in FIG. 1, a system 100 for controlling the motion of a joint 105 of a user includes a physiological sensor 115 adapted to measure a physiological property of the user, an orthosis 125 adapted to selectively assist and/or resist the motion of the joint of the user, an orthosis sensor 135 adapted to monitor the orthosis 125, and a processing unit 145 adapted to process data from the physiological sensor 115 and from the orthosis sensor 135. The motion of the joint 105 is preferably assisted and/or resisted to improve the mobility of the user or to improve a healing or rehabilitation process of the user. The joint 105 is preferably a ankle joint, but may alternatively be an elbow joint, a shoulder joint, a knee joint, a wrist joint, a hip joint, a neck joint, a back joint, a finger joint, a foot joint, or any other suitable joint. The motion of the joint 105 may alternatively or additionally be assisted or resisted to aid the post operative healing of a recently operated joint (such as a knee or hip replacement), the speed and quality of rehabilitation exercise (including gait training), and/or the healing of a broken bone. Aiding may include merely improving comfort (and may not actually include providing any measurable health benefits).

The physiological sensor 115 of the preferred embodiment functions to measure a physiological property of the user. The physiological sensor 115 is preferably a single sensor with multiple measurement functionalities; however, the physiological sensor 115 may include more than one sensor to measure different types of physiological responses from the user. The physiological sensor 115 preferably includes sensors to measure and collect physiological properties of the user, such as heart activity, blood pressure, blood oxygenation, and respiratory rate.

Heart rate, as well as other data derived from heart activity, is preferably measured as an electrocardiogram (ECG or EKG) with devices such as a Holter monitor (a long term heart monitor), an Event monitor (which constantly monitors heart activity, and a user presses a button when they feel symptoms and the monitor records information some period of time prior to the symptoms and some period of time after), an athletic heart monitor, or any other suitable heart monitoring device. To enhance the quality of heart rate data collection, wearable sensors integrated into clothing articles, such as a shirt containing heart monitoring leads, may be used, as taught in U.S. Pat. No. 7,324,841, which is incorporated in its entirety by this reference.

Preferably, blood pressure is measured without the use of actuated cuff mechanisms, with wearable blood pressure sensors such as those taught in US Application Nos. 2007/0055163 and 2008/0039731, and U.S. Pat. No. 6,554,773, which are all incorporated in their entirety by this reference. As taught in these references, such sensors are preferably optical-based blood pressure sensors that use adaptive hydrostatic calibration to estimate blood pressure from peripheral pulse transit time (PTF) measurements. Alternatively, blood pressure may be measured by any suitable blood pressure device.

Respiratory rate is preferably measured using a strain gauge band worn around a user's chest, but may alternatively be measured using breath sensors, a microphone, or other piezoelectric transducer, such as the F-SM and Model 1010 Microphones sold by Grass Technologies. These piezoelectric sensors are designed to reliably monitor and record peripheral pressure pulses, Korotoff sounds, carotid pulses, respiratory activity, swallowing, and other dynamic biological functions.

The physiological sensor 115 may include sensors to measure and collect alternative or additional physiological properties of the user such as temperature, brain waves, and perspiration rate. Temperature of the skin and/or body may be measured by a thermometer. Brain waves may be measured by a device taught in U.S. Pat. No. 6,572,558, which is incorporated in its entirety by this reference. Perspiration rate may be measured by water or humidity sensors, as taught in “Human perspiration measurement” Toshio Ohhashi et al 1998 Physiol. Meas. 19 449-461, which is incorporated in its entirety by this reference. Further, the physiological sensor 115 may include sensors to measure and collect alternative or additional physiological properties of the user such as oxygenation or any other suitable physiological property of the user.

The orthosis 125 of the preferred embodiment functions to assist and/or resist motion, while otherwise protecting, supporting, stabilizing, aligning, strengthening and/or improving a joint of the user. The orthosis 125 may also function to modify the motion of the joint by assisting, resisting, blocking, or unloading the body weight. The orthosis 125 is preferably a brace, more preferably a joint brace, but may alternatively be a brace, splint, sling, wrap, cast, or any other suitable orthosis 125. In one variation, the brace preferably includes a motor 155 to assist and/or resist joint motion. An example of a brace with a motor can be found in U.S. Ser. No. 11/932,799 filed on 31 Oct. 2007 and entitled “Methods and Devices for Deep Vein Thrombosis Prevention”, which is hereby incorporated in its entirety by this reference. Another example of a brace with a motor can be found in U.S. Ser. No. 12/191,837 filed on 14 Aug. 2008 and entitled “Actuator System with a Multi-Motor Assembly for Extending and Flexing a Joint”, which is also hereby incorporated in its entirety by this reference. In yet another alternative variation, the orthosis may be an immobilizing brace (such as a cast or a splint) that includes rigid elements to prevent joint motion or to resist joint motion.

The orthosis sensor 135 of the preferred embodiment functions to monitor the orthosis. Preferably the orthosis 125 is a brace and the orthosis sensor 135 functions to monitor a physical property of the brace such as flexion, extension, position, angle, force, speed, or acceleration of the portion (such as a joint) of the brace, or other suitable parameters. Alternatively, in the variation where the orthosis 125 is an immobilizing brace, such as splint, sling, wrap, cast, the orthosis sensor 135 may measure parameters such as a strain or pressure. In both variations, the orthosis sensor 135 may alternatively or additionally measure temperature within a layer of the orthosis 125, which could signal the relationship of the orthosis to the skin of the user (such as “too tight” or “not tight enough”). Further, in both variations, the orthosis sensor 135 may alternatively or additionally measure force or pressure exerted by the user, which—if located in a suitable location such as the soles of the feet of the user—could signal the weight distribution of the user.

The orthosis 125 of the preferred embodiment also includes a motor 155. The motor 155 functions to provide assistance and/or resistance to the joint of the user. The motor 155 may provide resistance (i.e. negative assistance) to strengthen the joint of a user, either adaptively (i.e. a selected motion of the joint), dynamically (i.e. selected portions of a repetitive joint motion or variable resistance or assistance based on a particular parameter), or constantly. The motor 155 preferably receives control instructions from the processing unit 145, which preferably controls the motion of the joint of a user based on the sensor data from the physiological sensor 115 and/or the orthosis sensor 135. In one variation, the processing unit 145 preferably controls the motor 155 to substantially maintain a physiological response of the user within a predetermined range of the targeted physiological response. In this variation, the processing unit may assist the user when the user is deemed to be under too much physical stress and may resist the user when the user is deemed to be under not enough physical stress.

The processing unit 145 of the preferred embodiment functions to process the sensor data from the orthosis sensor 135 and the physiological sensor 115 and to command the orthosis to assist or resist joint motion of the user (and/or to provide passive support). The processing unit 145 is preferably connected to the orthosis sensor 135 and the physiological sensor 115. The processing unit 145 is preferably located on the orthosis, but may be alternatively located in any suitable location (including in a remote location connected by any suitable communication channel, such as a mobile phone network). The physiological sensor 115 (and/or the orthosis sensor 135) and the processing unit 145 are preferably connected via a wired connection, but may alternatively be wirelessly connected. The wireless connection may be a cellular network connection, an 802.11 connection, a Bluetooth connection, a low power wireless connection (such as a connection between Nordic Semiconductor nRF24Lo1 or nRF24Lo1+ ultra low power wireless chipsets), or any other suitable communication connection.

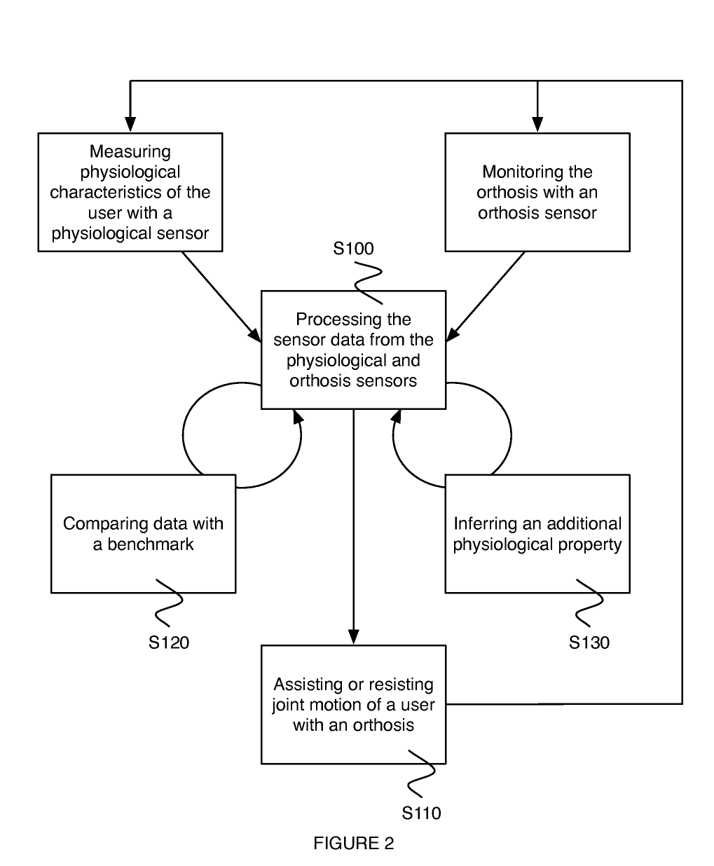

As shown in FIG. 2, a method for controlling the motion of a joint of a user includes the initial steps of measuring physiological characteristics of the user with the physiological sensor 115 and monitoring the orthosis with the orthosis sensor 135. After these initial steps, the method includes processing the sensor data from the physiological and orthosis sensors Step S100 and, based on the processed data, assisting or resisting joint motion of a user with an orthosis Step Silo. The method may include the additional steps of comparing data with a benchmark Step S120 and/or inferring an additional physiological property S130. The method preferably continues by returning to the initial state of measuring and monitoring.

The processing unit 145 is preferably adapted to process the sensor data from the orthosis sensor 135 and/or the physiological sensor 115 to determine or estimate physical exertion of the user. This determined physical exertion is preferably computed from brace motion of the user, but may alternatively be measured as a force (e.g. isometric exercise, muscular contractions, an impact from jumping), a frequency of a repetitive motion (e.g. a knee or ankle movement when walking), or any other suitable physical exertion.

In a first variation, the processing unit 145 uses the determined physical exertion as a substitute or a supplement for the data gathered in a conventional cardiac stress test (such as physical exertion data collected on a treadmill, exercise bike, or rowing machine). As used in this document, a cardiac stress test is a medical test performed to evaluate arterial blood flow to (and indirectly the amount of oxygen that will reach) the myocardium (heart muscle) during physical exercise, compared to blood flow while at rest. In this variation, the brace motors may be actively controlled by the processing unit 145 to provide resistance (or assistance) appropriate to the user with an orthosis S110, during the substitution or supplementation of the conventional cardiac stress test.

In a second variation, the processing unit 145 uses the determined physical exertion to perform an athletic fitness test. The orthosis sensor of this variation can monitor for pushups (if the orthosis is attached to the elbow or shoulder), which is part of the U.S. Army physical fitness test, or monitor for 2 om dashes (if the orthosis is attached to the knee or ankle), which is part of the shuttle run test. Both tests are used by sports coaches and trainers to estimate an athlete's VO2 max (maximum oxygen uptake) and ultimately estimate the cardio-vascular fitness of the athlete, which is one of the all important “Components of Fitness”. Like the first variation, the brace motors may be actively controlled by the processing unit 145 to provide resistance (or assistance) appropriate to the user with an orthosis S110, during the substitution or supplementation of the conventional athletic fitness test.

In a third variation, the data collected may be used to provide a rehabilitation program. In this variation, the physical exertion of the heart, lungs, and overall physique of the user during the rehabilitation can be measured and optimized, while providing optimal protection and nurtured healing of an injured joint (to be supported by the orthosis 125) during the rehabilitation This optimization is preferably based on the collected data. The assistance and/or resistance of the joint motion of the user S110 may be dynamically modified to maintain certain physical exertion levels based on the collected data. This variation may be used specifically for athletes to provide a balanced training/healing regimen.

In a fourth variation, the processing unit 145 generates an alarm signal when a physiological response of the user is outside a predetermined range of a targeted physiological response (such as a target heart rate). The processing may include comparing the data against historical data, demographic data, and/or training program target data, which is shown as Step S120 in FIG. 2. The processing performed by the processing unit may include providing visualizations of the data, recommendations of physical activity (such as exercise workouts), alerts (to the user, to a doctor, and/or to an emergency service such as “911”), information regarding the compliance of a particular rehabilitation regime, and/or medical record integration. For example, if a user is exercising too hard in their rehabilitation exercises (and the physiological response is greater than a predetermined threshold above a targeted physiological response), and the heart rate of the user surpasses a target level, an alarm signal may be generated. This alarm signal may be audible, visual, optical, electrical (shock), vibrational, pressurized, or in the form of assistance or resistance provided by the brace (i.e. a user now has a much easier or harder time moving their joint). Additionally or alternatively, the alarm signal may notify a medical professional such as a medical doctor, a rehabilitation specialist, a physical therapist, an emergency medical team member, a nurse's station, a family member or any other suitable person. The alarm signal may also be generated if the user is not exercising hard enough (and the physiological response is less than a predetermined threshold below a targeted physiological response heart rate of the user). This will alert the user that the training is not hard enough to achieve their workout goal and that they need to achieve higher physical exertion over a period of time. The alarm signal may be transmitted to a trainer (who may encourage or motivate the user to train with more vigor), or a training program to determine and adjust the optimum level of training for the user.

In a fifth variation, the processing unit 145 infers at least one additional physiological property from the data measured by the physiological sensor, which is shown as Step S130 in FIG. 2. The processing unit 145 may infer based on user demographics (e.g. age, weight, Body Mass Index), which may be computed from tables or calculated from numerical models. The model or table may be a simple thresholding, such as a calibrated breathing rate estimate for a resting heart rate of 60 and a calibrated breathing rate estimate associated with a target heart rate of 140. The inferred physiological property is preferably oxygenation. The inferred oxygenation of the user may be used to assess cardiovascular state and capability. In this variation, the orthosis brace may actively respond (via a motor 155 controlled by control signals from the processing unit 145) to the user's oxygenation level and reduce cardiovascular load by providing mobility assistance or increase cardiovascular load by providing mobility resistance. The inferred physiological property may, however, be any suitable physiological property, such as max heart rate, arrhythmia detection, target heart rate, and Heart Rate Variability (HRV), as taught in US Application No. 2007/0287928, which is incorporated in its entirety by this reference. In addition, physiological data such as the respiration rate and EPOC (excess post-exercise oxygen consumption) may be computed from the measured heart rate, which is taught by Firstbeat and Suunto in U.S. Pat. No. 7,192,401, US Application No. 2006/0004265, and US Application No. 2007/0265534, which are all incorporated in their entirety by this reference.

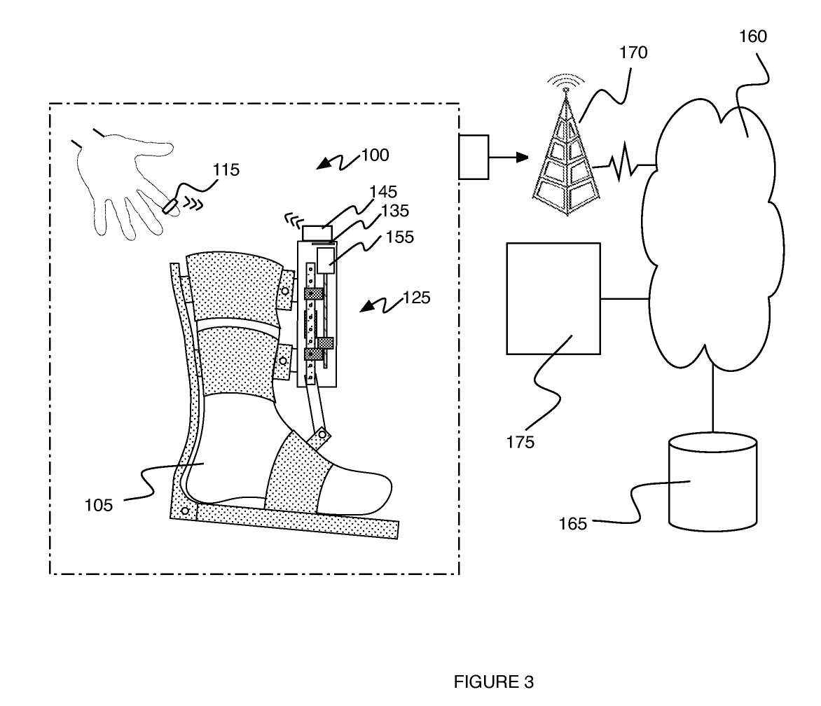

In a sixth variation, as shown in FIG. 3, the processing unit 145 uploads information through the Internet 160 to a remote server 165. The information may include data from the physiological sensor 115, data from the orthosis sensor 135, determinations, calculations, or estimates by the processing unit 145, or any other suitable information. The information on the remote server is preferably accessible by a medical professional via a suitable electronic device 175 (either directly or through any other suitable methods, such as electronic mail) and is preferably used to review the user's recovery, motion habits, health, cardiac stress, compliance to a particular rehabilitation regime, or any other suitable data from the uploaded information. The information upload is preferably performed over a connection to a digital cellular (such as a GPRS) tower 170, but may alternatively be performed over a USB connection, a Bluetooth connection, a wireless LAN connection, a serial port connection, a digital media card (SD card) inserted into a card slot, an Ethernet connection, a digital or analog modem, infra red connection, or any other suitable connection.

In a seventh variation, the processing unit 145 is adapted to download software for communicating with a different physiological sensor. For example, a commercial athletic heart rate monitor, or a medical holter monitor, could interface with the processing unity 145 after downloading a suitable device driver to enable communication with the particular physiological sensor. This enables the use of additional or alternative sensors as physiological sensor technology advances. The software download is preferably performed over a connection to a digital cellular (such as a GPRS) tower 170 through the Internet 160, but may alternatively be performed over a USB connection, a Bluetooth connection, a wireless LAN connection, a serial port connection, a digital media card (SD card) inserted into a card slot, an Ethernet connection, a digital or analog modem, infra red connection, or any other suitable connection.

In an eighth variation, the processing unit 145 is adapted to generate gait and/or weight distribution data from an orthosis sensor located near the feet of the user (such as sensors embedded within a shoe insert). This data is preferably compared to other baseline data to assess changes and respond accordingly. Deviations may indicate susceptibility to a fall or injury due to loss of balance, may indicate muscular or skeletal conditions, or may indicate an asymmetric gait of the user. This data may alternatively be used to assess the gait and weight distribution improvement of a post-stroke patient, and may provide feedback to assess neuro-muscular recovery and control. In this variation, the system may respond with assistance, resistance, or a warning signal (such as a vibration or an audible signal).

In a ninth variation, the processing unit 145 includes every suitable permutation and combination of the previous eight variations.

As a person skilled in the art will recognize from the previous detailed description and from the figures and claims, modifications and changes can be made to the preferred embodiments of the invention without departing from the scope of this invention defined in the following claims.

I claim:

1. An apparatus for controlling the motion of a joint of a user, comprising:
a physiological sensor that measures a physiological property of the user;
an orthosis that selectively modifies the motion of the joint of the user;
an orthosis sensor that monitors a physical property of the orthosis; and
a processing unit that processes data from the physiological sensor and from the orthosis sensor.

2. The apparatus of claim 1, wherein the orthosis is selected from the group consisting of: splint, cast, sling, and wrap.

3. The apparatus of claim 2, wherein the orthosis sensor monitors strain.

4. The apparatus of claim 1, wherein the orthosis is a brace.

5. The apparatus of claim 4, wherein the orthosis sensor monitors a physical property of the brace selected from the group consisting of: flexion, extension, position, angle, force, speed, or acceleration, strain, temperature, and pressure.

6. The apparatus of claim 5, wherein the processing unit processes data from at least one of the orthosis sensor and physiological sensor to determine physical exertion of the user.

7. The apparatus of claim 6, wherein the processing unit processes data from the physiological sensor and the orthosis sensor to at least supplement a cardiac stress test.

8. The apparatus of claim 6, wherein the processing unit processes data from the physiological sensor and the orthosis sensor to perform an athletic fitness test.

9. The apparatus of claim 6, wherein the brace selectively provides assistance to the motion of the joint of the user, and wherein the brace selectively provides resistance to the motion of the joint of the user.

10. The apparatus of claim 9, wherein the processing unit controls the assistance and the resistance of the motion based on data from the physiological sensor and from the orthosis sensor.

11. The apparatus of claim 10, wherein the processing unit controls the assistance and the resistance to substantially maintain a targeted physiological response of the user.

12. The apparatus of claim 10, wherein the processing unit generates an alarm signal when a physiological response of the user is outside a predetermined range of the targeted physiological response.

13. The apparatus of claim 1, wherein the processing unit compares data from the physiological sensor against at least one of the following: physiological properties of the user in the past, demographic averages of physiological properties of other users, and targeted physiological properties for the user.

14. The apparatus of claim 1, wherein the measured physiological property is selected from the group consisting of: heart rate, blood pressure, oxygenation, respiratory rate, brain waves, temperature, and perspiration rate.

15. The apparatus of claim 1, wherein the processing unit infers a physiological property based on data from the physiological sensor.

16. The apparatus of claim 15, wherein the inferred physiological property is selected from the group consisting of: heart rate, blood pressure, oxygenation, respiratory rate, brain waves, and perspiration rate.

17. The apparatus of claim 1, wherein the physiological sensor and the processing unit are wirelessly connected.

18. The apparatus of claim 1, wherein the orthosis sensor and the processing unit are wirelessly connected.

19. The apparatus of claim 1, further comprising a transmitter adapted to upload data from the physiological sensor to a remote server.

20. The apparatus of claim 19, wherein the uploaded data on the remote server is accessible by a medical professional.

21. The apparatus of claim 1, wherein the processing unit is adapted to download software for communicating with, and processing data from, a different physiological sensor.

22. A method for controlling the motion of a joint of a user, comprising the steps of:
measuring a physiological property of the user with a physiological sensor;
selectively modifying motion of the joint of the user with an orthosis;
monitoring a physical property of the orthosis with an orthosis sensor; and
processing data from the physiological sensor and from the orthosis sensor.

23. The method of claim 22, wherein the orthosis is a brace, and the step of monitoring the orthosis includes determining a physical property of the brace.

24. The method of claim 23, wherein the step of monitoring the orthosis includes monitoring a physical property of the brace selected from the group consisting of: flexion, extension, position, angle, force, speed, acceleration, strain, temperature, and pressure.

25. The method of claim 23, further comprising the step of determining physical exertion of the user.

26. The method of claim 25, wherein the step of determining physical exertion includes processing data from the orthosis sensor.

27. The method of claim 25, further comprising the step of processing the determined physical exertion and data from the physiological sensor to at least supplement a cardiac stress test.

28. The method of claim 25, further comprising the step of processing the determined physical exertion and the data from the physiological sensor to perform an athletic fitness test.

29. The method of claim 23, further comprising the step of selectively providing assistance of the motion of the joint of the user, and selectively providing resistance of the motion of the joint of the user.

30. The method of claim 29, further comprising the step of processing of the determined physical exertion and data from the physiological sensor to determine the amount of assistance and resistance.

31. The method of claim 30, wherein the step of providing assistance and resistance includes providing assistance and resistance to substantially maintain a targeted physiological response of the user.

32. The method of claim 30, further comprising the step of generating an alarm signal when a physiological response of the user is outside a predetermined range of the targeted physiological response.

33. The method of claim 30, further comprising the step of comparing data from the physiological sensor against at least one of the following: physiological properties of the user in the past, demographic averages of physiological properties of other users, and targeted physiological properties for the user.

34. The method of claim 22, wherein the measured physiological property is selected from the group consisting of: heart rate, blood pressure, oxygenation, respiratory rate, brain waves, temperature, and perspiration rate.

35. The method of claim 22, wherein the step of measuring a physiological property includes inferring a physiological property from data from the physiological sensor.

36. The method of claim 35, wherein the inferred physiological property is selected from the group consisting of: heart rate, blood pressure, oxygenation, respiratory rate, brain waves, and perspiration rate.

37. The method of claim 22, further comprising the step of uploading data from the physiological sensor to a remote server.

38. The method of claim 37, further comprising allowing access of the data from the physiological sensor on the remote server.

39. The method of claim 22, further comprising the step of downloading software for a different physiological sensor.
